(19) United States
(12) Patent Application Publication
(10) Pub. No.: US 2023/0037138 A1
(43) Pub. Date: Feb. 2, 2023

(54) PARAXANTHINE-BASED CAFFEINE SUBSTITUTE COMPOSITIONS AND METHOD OF USE THEREOF IN SLOW CAFFEINE METABOLIZERS

(71) Applicant: Ingenious Ingredients, LP, Lewisville, TX (US)

(72) Inventors: Ralf Jäger, Whitefish Bay, WI (US); Martin Purpura, Spring, TX (US); Shawn D. Wells, Frisco, TX (US); Kylin Liao, Plano, TX (US)

(21) Appl. No.: 17/875,368

(22) Filed: Jul. 27, 2022

Related U.S. Application Data
(60) Provisional application No. 63/226,113, filed on Jul. 27, 2021.
(60) Provisional application No. 63/331,732, filed on Apr. 15, 2022.

(51) Int. Cl. A61K 31/522; C12Q 1/6883; A61P 25/00; A61K 31/197; A61K 31/185; A61K 31/439; A61K 31/205; A61K 31/7068; A61K 31/661; A61K 31/714; A61K 31/047; A61K 31/155; A61K 36/258

(52) U.S. Cl. CPC A61K 31/522; A61K 31/047; A61K 31/155; A61K 31/185; A61K 31/197; A61K 31/205; A61K 31/439; A61K 31/661; A61K 31/714; A61K 31/7068; A61K 36/258; A61P 25/00; C12Q 1/6883; C12Q 2600/156

(57) ABSTRACT

The disclosed compositions methods relate to a dietary supplement for subjects with slow caffeine metabolism and comprises paraxanthine and optionally other compounds that modulate the effects of paraxanthine. Uses for the paraxanthine-containing supplements contain improvement of at least one of endurance performance, mood, vigor, lipolysis, energy expenditure, exercise performance, and/or decreased appetite in a slow caffeine metabolizer subject.

### CROSS-REFERENCE TO RELATED APPLICATION(S

This application claims the benefit under 35 U.S.C. § 119(e) to U.S. Provisional Application 63/226,113, filed Jul. 27, 2021, and entitled “CAFFEINE SUBSTITUTES HAVING REDUCED BITTERNESS AND METHOD OF USE THEREOF,” and to U.S. Provisional Application 63/331,732, filed Apr. 15, 2022, and entitled “PARAXANTHINE-BASED BIOACTIVE COMPOSITION AND METHOD OF USE THEREOF IN SLOW CAFFEINE METABOLIZERS,” each of which is hereby incorporated herein by reference in its entirety for all purposes.

### TECHNICAL FIELD

The disclosed technology relates generally to compositions, methods, and system for utilizing paraxanthine alone and in combination for use in providing physiological benefits to certain subjects who metabolize caffeine slowly. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Caffeine is a bitter, white crystalline purine, a methylxanthine alkaloid, and is chemically related to the adenine and guanine bases of deoxyribonucleic acid (DNA) and ribonucleic acid (RNA). It is found in the seeds, nuts, or leaves of several plants native to Africa, East Asia and South America, and helps to protect them against predator insects and to prevent germination of nearby seeds. The most well-known source of caffeine is the coffee bean, a misnomer for the seed of Coffea plants.

Caffeine concentrations in coffee beverages can be quite variable. A standard cup of coffee is often assumed to provide 100 mg of caffeine, but a recent analysis of 14 different specialty coffees purchased at coffee shops in the US found that the amount of caffeine in 8 oz (~240 ml) of brewed coffee ranged from 72-130 mg (McCusker, R. R., Goldberger, B. A. and Cone, E. J. 2003. Caffeine content of specialty coffees. J. Anal. Toxicol., 27:520-522.). Caffeine in espresso coffees ranged from 58-76 mg in a single shot. Interestingly, the caffeine content of the same type of coffee purchased from the same store on six separate days varied from 130 to 282 mg per 8-oz serving.

Furthermore, there is substantial variability within the population in how individuals respond to caffeine. This is function of variability of the way caffeine is metabolized between individuals. Roughly speaking, the population can be divided into three groups:
1. Fast metabolizers: individuals that metabolize caffeine quickly that they experience little to no effects from caffeine itself. This group tends to be able to sleep shortly after consuming caffeine. They experience few side effects but also few benefits.
2. Average metabolizers: individuals for whom the experience benefits/energy of caffeine without being overly burdened by side effects.
3. Slow metabolizers: individuals for whom caffeine is metabolized at a significantly slower rate than the population on average. Such individuals may have side effects of 12-24 hours in length with brain fog, headaches, arrhythmia, tachycardia, hypertension, sleep disturbance, among others. For these individuals, any benefit from caffeine is often outweighed by the many of side effects they experience.

Thus, there is a need in the art to identify alternative chemical compounds and mixtures thereof that may provide the benefits of caffeine that slow metabolizers are otherwise unable to enjoy. It is also desirable to provide chemical compounds and mixtures thereof that may be used to provide a variety of benefits, varying by concentration, thus requiring production of fewer materials.

### BRIEF SUMMARY

Disclosed herein are compositions and methods for improving cognitive function in a slow caffeine metabolizer (SCM) subject by identifying an individual as a SCM subject; and providing the SCM subject with a composition comprising paraxanthine. In certain embodiments, the step of identifying the SCM subject comprises genotyping the individual for a genetic variant associated with slow caffeine metabolism. In certain implementations, the genetic variant is in a CYP1A2, ADORA2A and/or CYP2E1gene. In exemplary implementations, the genetic variant is in the CYP1A2 gene and the subject’s genotype is AC or CC. In certain embodiments, the SCM subject is identified by administering to the individual a questionnaire on the individual’s response to caffeine. In further embodiments, the SCM subject self-diagnoses as SCM. In yet further embodiments, the SCM subject is identified by measuring the individual’s rate of caffeine metabolism.

In certain embodiments, improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition in the SCM subject. According to certain embodiments, administration of the composition to the SCM subject increases serum Brain Derived Neurotrophic Factor (BDNF) concentration of at least about 10% relative to a fast caffeine metabolizer subject who received a comparable dose of the composition.

In certain embodiments, the composition further comprises at least one agent selected from 1-methylxanthine and/or 7-methylxanthine. In exemplary implementations, 1-methylxantine and/or 7-methylxantine are present in the composition in an amount from about 50 mg to about 400 mg. According to further embodiments, the composition further comprises at least one ingredient selected from the group consisting tyrosine, N-acetyl-tyrosine, taurine, huperzine A, acetyl-1carnitine, CDP choline, Alpha GPC, choline bitrate, choline citrate, B12, caffeine, methyllliberine, theacrine, paraxanthine, theobromine, ashwagandha, rhodiola, lutein, zeaxanthin, fish oil, creatine, ginseng, lions mane, niacin, cordyceps, theanine, B-vitamins, GABA, sulbutiamine, vinpocetine, adenosine triphosphate, inositol, enhanced arginine silicate, nitrates, electrolytes, hesperidin and derivatives of hesperidin and/or bacopa.

Further disclosed herein is a method for improving athletic performance in a SCM subject in comprising administering to the subject a composition comprising an effective amount of paraxanthine. In certain implementations, the amount of paraxanthine is administered is from about 50 mg to about 400 mg.

In certain embodiments, the subject experiences and increase in endurance.

In certain embodiments, the composition further comprises 1-methylxanthine in an amount from about 2 mg to about 800 mg and wherein administration of the composition to a subject produces a synergistic increase in athletic performance to the administration of a comparable dose of paraxanthine or 1-methylxanthine alone.

In further embodiments, administration of the composition to the SCM subject increases serum N-methylnicotinamide (MNA) concentration from about 20-100% relative to the change in the subject following administration of a comparable dose of caffeine.

Further disclosed herein is a method of promoting weight loss in a SCM subject comprising by identifying an individual as a SCM subject; and providing the SCM subject with a composition comprising about 25 mg to about 400 mg of paraxanthine. In certain embodiments, weight loss is promoted through inducing thermogenesis in the subject.

In certain implementations of these embodiments, the composition further comprises one or more compounds selected from a list consisting of: caffeine, green tea, capsaicin, garcinia cambogia, yohimbine and bitter orange.

In further embodiments, administration of the composition to the SCM subject increases serum N-methylnicotinamide (MNA) concentration from about 20-100% relative to the change in the subject following administration of a comparable dose of caffeine and wherein the subject experiences an increased perception of energy and/or a decreased perception of jitteriness. In further embodiments, weight loss is promoted through enhancing lipolysis in the subject.

In certain embodiments, the disclosed compositions comprise congeners, derivatives and iterations of paraxanthine and synthetic chemical equivalents of paraxanthine.

According to certain embodiments, the disclosed compositions comprise agglomerated paraxanthine, as salts, microencapsulated, liposomal or esterified versions of them.

According to certain embodiments, paraxanthine is combined with glycerides, propylene glycol, polyethylene glycol (PEG), lauroyl macrogol, lauroyl macrogol derivatives and co-crystallization products of 1-methylxanthine and paraxanthine.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “decaffeinated product” refers to a product, which typically contains caffeine, that has some, most or all of the caffeine removed (e.g., via a decaffeination process). For example, the decaffeinated product can have a reduced amount of caffeine (e.g., 95% removal, 97% removal, 99% removal) compared to the amount normally associated with that product.

In certain embodiments, the decaffeinated product is a coffee product. In further embodiments, the decaffeinated product is tea product.

In yet further embodiments, the decaffeinated product is a soft drink or an energy drink, or the like. In these embodiments, it is not necessary for a decaffeination process to occur, rather caffeine is simply reduced or omitted as an ingredient.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is substantially free of particles would either completely lack particles, or so nearly completely lack particles that the effect would be the same as if it completely lacked particles. In other words, a composition that is substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

As used herein, a “slow caffeine metabolizer” also referred to herein as “SCM” is a subject whose metabolism of caffeine is substantially slower than the median rate of metabolism of the general population. In certain embodiments, SCM subjects are defined as subjects having a (C/A) single nucleotide polymorphism at intron 1 of the cytochrome P450 (CYP1A2) gene. Specifically, individuals who are homozygous C/C for this polymorphism. Additional genetic markers for SCM are described by Thorn CF, Et al. PharmGKB Summary: Caffeine Pathway. PHARMACOGENET GENOMICS. (2012);22(5):389-395, which is incorporated by reference in its entirety.

SCM individuals may make up between 30-60% of the population. In certain, a person may be an SCM for reasons other than genetic factors. For example, alcohol and grapefruit juice both have the effect decreasing caffeine clearance rates. Similarly, oral contraceptives decrease the rate of caffeine metabolism, particularly during the second half of the menstrual cycle. Furthermore, pregnancy, particularly during the third trimester decreases the rate of caffeine metabolism. Accordingly, the composition and method disclosed herein can be administered, with beneficial effects, to subjects with SCM attributable to either genetic or non-genetic factors.

Disclosed are compositions comprising paraxanthine and the related uses thereof. Paraxanthine is also known as 1,7-dimethylxanthine or 1,7-dimethyl-3H-purine-2,6-dione. Paraxanthine may be produced synthetically or may be isolated from a natural source (e.g., extraction) or through fermentation. Paraxanthine isolated from such sources may be purified to 95% or greater purity. Optionally, less purification may be used such that paraxanthine accounts for 50%, or even less, of the material. In some embodiments, it may be preferable to utilize paraxanthine isolated from a natural source which may include other congeners of paraxanthine typically found in paraxanthine sources.

In certain alternative embodiments, the disclosed compositions comprise paraxanthine congeners or analogs. In certain, the paraxanthine congener or analog is 1-methylxanthine, 3-methylxanthine, and/or 7-methylxanthine. In certain embodiments the composition comprises one of the foregoing methylxanthines. In further embodiments, the composition comprises a combination of the foregoing methylxanthines. In yet further embodiments, the composition comprises one ore more methylxanthines and paraxanthine.

In certain embodiments, paraxanthine may be combined with one or more other chemical compounds (e.g. other active ingredients), to provide a plurality of positive effects in a subject. By altering the dosage of paraxanthine and/or chemical compounds it is combined with, various physiological effects may be selected for. The compositions may provide primarily a single benefit, or may provide multiple benefits simultaneously. In certain embodiments, paraxanthine is combined with one or more additional active ingredients selected from: a group consisting of: gallic acid, (+)-catechin (C), (-)- epicatechin (EC), (+)-gallocatechin (GC), (-)-epigallocatechin (EGC), (-)-catechin gallate (CG), (-)-gallocatechin gallate (GCG), (-)-epicatechin gallate (ECG) and (-)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (Sadenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, ginkgo biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(1), Nicotinamide riboside, picamilon, Huperzine A (Chinese clubmoss or Huperzia serrata, L-dopa, Mucuna pruriens, and forskolin (Coleus forskohlli), 2- (dimethylamino)ethanol (DMAE), DMAE bitartrate, and combinations thereof.

In another embodiment, paraxanthine may be used at lower dosage levels and/or in conjunction with compounds that modulate or antagonize its activity. Such compositions may induce an improved endurance performance, mood, vigor, lipolysis, energy expenditure, exercise performance, and/or decreased appetite.

An advantage of using the invention may be the reduced likelihood that a slow caffeine metabolizer subject develops a tolerance to chemical compositions in accordance with the principles of the invention. That is, a person may not become desensitized to the effects induced.

According to certain aspects, the disclosed paraxanthine containing compositions has at least the following distinct advantages over the administration of compositions containing comparable doses of caffeine in slow caffeine metabolizer subjects. Paraxanthine has substantially lower toxicity, especially for slow caffeine metabolizer subjects. Paraxanthine has greater stability (e.g. does not lose potency over time to the same extent as caffeine). Paraxanthine containing compositions are more potent wake-promoting agent (in certain embodiments, via adenosine receptor antagonism). Further, paraxanthine containing compositions enhance striatal dopaminergic tone. Still further, paraxanthine does not produce sleep rebound. Further, paraxanthine does not produce withdrawal effects upon cessation of use, as frequently occurs with caffeine. Yet further, paraxanthine does not enhance anxiety. Still further, paraxanthine is less bitter than caffeine. In another embodiment, paraxanthine may be used at higher dosage levels and/or with synergistic compounds.

These compositions may increase a slow caffeine metabolizer subject’s basal/resting metabolic rate, increase thermogenesis, decrease appetite, enhance cognitive performance, increase alpha wave brain activity, and/or induce euphoria. Without being bound by theory, the inventors believe that at higher dosage levels, paraxanthine may be noradrenergic and dopaminergic, and may exhibit increased adenosine receptor inhibition.

In another embodiment, paraxanthine is combined with ephedrine, caffeine, salicylic acid or the like. The foregoing combinations may produce a synergistic effect with the stimulating effects of paraxanthine in a SCM subject. For example, in certain embodiments, paraxanthine is combined with much lesser amounts of caffeine in order to modulate the excessive stimulatory effects of caffeine, thereby stabilizing heart rate and other metabolic activity. That is, a combination of paraxanthine and caffeine may result in a composition that imparts the increased focus and energy induced by caffeine, but without the higher heart rate and blood pressure due to modulation of caffeine’s effects by paraxanthine. Thus the combination (or paraxanthine alone) may result in heightened awareness and calmness without the jitters caffeine that caffeine causes in SCM subjects.

A further advantage of the instant disclosure is administering paraxanthine based compositions avoid exposing SCM subjects to theobromine and Theophylline. Theobromine and Theophylline are two caffeine metabolites which may contribute to the adverse effects of caffeine consumption. In particular theophylline-a controlled substance and bronchodilator-has a much greater toxicity than caffeine or any of its other metabolites. Its side effects include arrhythmia, confusion, increased blood pressure, anxiety, and sleep disturbance. CYP1A2 and CYP2E1 are both involved in conversion of theophylline and theobromine to their metabolites (xanthine and methyluric acid), and thus, slow metabolizers may have longer acting theophylline and theobromine, than fast metabolizers. Accordingly, SCM subjects may experience caffeine, theobromine, and theophylline all exerting physiologic effects and side effects for 12-24 hours, whereas fast metabolizers do not. Administration of paraxanthine to a SCM subject avoids the pathway that yields these metabolites, resulting in a cleaner experience of energy to the user.

According to further embodiments, dietary supplements comprising paraxanthine are used to enhance athletic performance. According to exemplary aspects of these embodiments, may be provided to a SCM subject in order to reduce fatigue, improve energy, increase mobility, and improve alertness. In further embodiments, administration of the disclosed compositions to a SCM subject is cardio protective. In further embodiments, administration of the disclosed compositions improves muscle contractions and muscle performance in a SCM subject. In exemplary aspects, of these embodiments, muscle performance is enhanced through increasing potassium (K+) transport into skeletal muscle. In further aspects, muscle performance is enhanced through increasing intracellular calcium (e.g., via ryanodine receptor (RyR) activation).

In another embodiment, paraxanthine may be used as a topical agent for incorporation into body creams or lotions to produce a cream or lotion for lightening skin, firming skin, and/or improving skin elasticity. A paraxanthine topical agent may also be used to promote localized transdermal fat loss. Paraxanthine may also be used in a cream or lotion to promote localized enhanced metabolism and/or enhanced thermogenesis.

According to further embodiments, paraxanthine is be combined with one or more of analgesics and/or anti-inflammatory agents. In exemplary implementations, paraxanthine is combined with ibuprofen, salicylic acid, anti-inflammatory agents, salicin, fish oil (omega-3 fatty acids and specialized, small lipid pro-resolving derivatives), tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate and/or triterpenoids.

The dosage of paraxanthine may range from about 2 mg to about 800 mg. In another embodiment, the range may be from about 50 mg to about 400 mg. In another embodiment, paraxanthine is combined with one or more bioavailability enhancers. In exemplary embodiments, bioavailability enhancers include, but are not limited to: bioperine, piperine, black pepper, bergamottin, dihydroxybergamottin (CYP3A4 inhibitors), flavonoids (including hesperidin, naringin, tangeritin, quercetin and nobiletin both in isolation and in combination), pterostilbenes, fisetin, nanoencapsulation, microencapsulation, liposomes and/or phytosomes. The enhancers that are combined with paraxanthine may depend on which qualities of paraxanthine are desired for a particular use.

In another embodiment, paraxanthine may be administered to a SCM subject using one or more delivery methods, including, for example transdermal patches and/or creams, ready to mix powders, intravenous methods, capsules, tablets, liquid (including liquids for mixing with other beverages), softgels, shot format, and/or cosmetic applications including soaps, lotions and shampoos. paraxanthine’s anti-inflammatory qualities may be desired for a variety of topical applications.

### Methods of Use

Further disclosed herein is a method for improving physical performance or energy in a slow caffeine metabolizer (SCM) subject by identifying an individual as a SCM subject and providing the SCM subject with a composition comprising about 2 mg to about 800 mg of paraxanthine. In certain embodiments, the step of identifying the SCM subject involves genotyping the individual for a genetic variant associated with slow caffeine metabolism.

The term “genotype” refers to the specific allelic composition of an entire cell or a certain gene, whereas the term “phenotype” refers to the detectable outward manifestations of a specific genotype.

As used herein, “genotyping” a subject (or DNA sample) for a polymorphic allele of a gene (s) refers to detecting which allelic or polymorphic form(s) of the gene(s) are present in a subject (or a sample). As is well known in the art, an individual may be heterozygous or homozygous for a particular allele. More than two allelic forms may exist, thus there may be more than three possible genotypes.

In exemplary implementations, the genetic variant is in the CFP1A2, ADORA2A and/or CYP2E1 genes. In further exemplary implementations, the individual is identified as a SCM subject if the individual carries a CYP1A2*1F variant. In certain embodiments, the genetic variant is in the CYP1A2 gene and the subject’s genotype is AC or CC.

According to further embodiments, the SCM subject is identified by administering the individual a questionnaire on the individual’s response to caffeine. In these embodiments, the individual may be asked various questions such as daily caffeine consumption, side effects experienced after caffeine consumption, and the effect of caffeine on sleep in individual. Those skilled in the art will appreciate that other areas of inquiry are possible in identifying SCM subjects. In certain implementations, the individual may self-identify as a SCM. In such implementations, the individual may be guided in self-identification through an online questionnaire or the like. Further implementations, involve informing the individual of one or more features that characterize SCM subjects.

According to still further embodiments, identifying SCM subjects may further comprise measuring an individual’s rate of caffeine metabolism. Such steps may be performed by administering caffeine to an individual and assaying the blood or urine for the presence of caffeine metabolites at various time points after administration.

According to certain embodiments, the composition disclosed herein are used in the treatment of one or more medical conditions in a SCM subject in need thereof. In certain implementations, the disclosed composition is administered to a SCM subject suffering from narcolepsy, sleep apnea, and shift work sleep disorder, insomnia epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson’s disease, Alzheimer’s, and/or dementia.

In certain aspects, the disclosed compositions are a neuroprotective agent. In certain embodiments, administration of the disclosed compositions to a SCM subject in need thereof is neuroprotective. In exemplary aspects of these embodiments, this neuroprotection is in the form of protecting against dopaminergic cell death.

Depending upon the subject to be treated and the route of administration, the compounds of the invention may be administered at varying doses. Although doses will vary from subject to subject, suitable daily doses paraxanthine are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses.

Advantageously, compositions of the present disclosure may be administered in single doses, e.g. once daily or more seldom, or in a total daily dosage administered in divided doses of two, three or four times daily. In certain embodiments, the composition is administered as needed (e.g., when the subject is in need of enhance energy, athletic or cognitive performance or the like).

### Athletic Performance

Further disclosed herein is a method for enhancing performance or energy in a SCM subject, comprising administering to the subject a composition disclosed herein. As used herein the term “enhancing performance” is intended to mean any improvement in performance. Performance can be assessed in any manner. Certain enhancements are readily measured. For example, in a timed-event, an improved time can assess an enhanced performance. Certain performance enhancing properties can be judged subjectively by the athlete or performer or an observer. In these instances, an enhanced performance means that the performance was perceived subjectively to be improved, magnified, faster, better and the like. In certain embodiments, the disclosed methods are used to enhance athletic performance. “Athletic performance” refers to any professional or recreational activity wherein the performer, for example an athlete, exerts a physical act, such as running, swimming, golf, bowling, archery, football, baseball, basketball, soccer, hiking, cycling, dancing and the like. In certain athletic performance is improved through in improvement of endurance in the subject. In other words, administration of the disclosed compositions improves a subject’s level of endurance, thereby enhancing the subject’s athletic performance. In further embodiments, administration of the composition to the subject increases cognitive performance which thereby improves athletic performance.

In certain embodiments, upon administration of the composition, the SCM subject experiences improvement of at least one of mood, energy, focus, concentration or sexual desire or a reduction of at least one of anxiety, fatigue, perception of effort or perception of pain.

In further embodiments, upon continued administration to the subject, the composition does not create dependence in the subject and/or withdrawal effect in the subject when continued use is ceased.

Further disclosed herein is a method of increasing athletic endurance in a SCM subject comprising administering to the subject a composition disclosed herein. In certain implementations, the composition administered to the subject comprises 1-methylxanthine and paraxanthine. In exemplary implementations, the administration of paraxanthine and 1-methylxanthine produce a synergistic increase athletic endurance in the subject, relative to the administration of paraxanthine or 1-methylxanthine alone.

According to further embodiments, administration of the disclosed composition to the subject increases the subject’s perceived level of energy. In exemplary implementations, the subject experiences an increase in energy of at least about 5 percent. According to certain embodiments, the composition administered further comprises (in addition to 1-Mx and/or paraxanthine) at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, huperzine A, theacrine, methylliberine, B12, sulbutiamine, magnolia bark, ketones, MCTs, omega 3's, lutein, zeaxanthin, tyrosine and n-acetyl-tyrosine, taurine, acetyl-1-carnitine and/or combinations thereof.

In certain embodiments, the SCM subject’s perceived level of energy is increased by between about 2% and about 50%. In further embodiments, the subject’s perceived level of energy is increased by between about 5% and about 30%. In yet further embodiments, the subject’s perceived level of energy is increased by between about 10% and about 25%.

N-methylnicotinamide (MNA) is a novel myokine that enhances the utilization of energy stores in response to low muscle energy availability (Ström et al. Changes in MNA levels are associated with an increase in energy, improved weight management and increased fat burning/loss. In certain implementations, administration of the disclosed composition increases serum MNA concentration from about 30-70% relative to SCM subjects who received placebo. In further embodiments, administration of the disclosed composition increases serum MNA concentration about 50% relative to SCM subjects who received placebo. In yet further embodiments, administration of the disclosed composition increases serum MNA concentration of at from about 20-50% relative to fast caffeine metabolizers who received a comparable dose of the composition. In yet further embodiments, administration of the disclosed composition increases serum SCM concentration about 75% relative to SCM subjects who received caffeine.

### Muscle Function

Further disclosed herein is a method for increasing muscle function in a subject by administering to the subject a composition disclosed herein. In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

According to certain embodiments, compositions disclosed herein may be administered in conjunction with a strength training regime. As will be appreciated by a person having skill in the art, administration of effective amounts of the disclosed compositions results in improved strength and improved athletic performance/ergogenesis in the subject.

In one aspect, the disclosed compounds inhibit muscle atrophy. In a further aspect, the disclosed compounds increase muscle mass. In a still further aspect, the disclosed compounds induce muscle hypertrophy. In a yet further aspect, the disclosed compounds inhibit of muscle atrophy and increase muscle mass. In an even further aspect, the disclosed compounds inhibit of muscle atrophy and induce muscle hypertrophy. In a further aspect, the inhibition of muscle atrophy is in a subject. In an even further aspect, the increase in muscle mass is in a subject. In a still further aspect, the subject is a mammal. In a yet further aspect, the mammal is a human.

In certain aspects, administration of the disclosed compositions is effective at preventing or treating age-related muscle atrophy or sarcopenia. In further aspects, administration of the disclosed compositions is effective at preventing or treating muscle atrophy associated with muscle immobilization, such as that which frequently occurs with casting of fractured bones. In further aspects, administration of the disclosed compositions is effective at preventing or treating muscle atrophy associated with disease, such as cancer, also known as cachexia.

According to certain aspects the composition is administered to a subject that has sarcopenia. In various aspects, the composition is administered in a therapeutically effective amount. In further aspects, the composition is administered at prophylactically effective amount, (e.g. to a subject at risk for developing sarcopenia, cachexia, or immobilization induced atrophy).

In certain aspects, the composition further comprises one or more additional active ingredient to further enhance muscle strength, size, and/or muscle function. In certain embodiments, the one or more additional active ingredient is an amino acid. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, ergothioneine, and tyrosine. According to the certain embodiments, the conditionally essential amino acid is tyrosine. In still further embodiments, the amino acid is selected from the group of non-essential amino acids including, but not limited to, alanine, aspartic acid, asparagine, glutamic acid, serine, selenocysteine and pyrrolysine. In yet further embodiments, the amino acid derivative is selected from the group of creatine, carnitine, beta-alanine, taurine, beta-hydroxy beta-methylbutyrate L-Arginine, omega-3 fatty acids, Vitamin D, whey protein, BAIBA, and other protein extracts from animal, plant or fermentation sources.

According to exemplary aspects of these embodiments, that may reduce fatigue, improve energy, increase mobility, and improve alertness. In further embodiments, administration of the disclose compositions is cardio protective. In further embodiments, administration of the disclose compositions improves muscle contractions and muscle performance. In exemplary aspects, of these embodiments, muscle performance is enhanced through increasing potassium (K+) transport into skeletal muscle. In further aspects, muscle performance is enhanced through increasing intracellular calcium (e.g., via ryanodine receptor (RyR) activation).

In certain aspects of the foregoing embodiments wherein the composition comprises effective amounts of 1-methylxanthine and paraxanthine, the administration of paraxanthine and 1-methylxanthine produce a synergistic increase in muscle size and/or function in the subject, relative to the administration of paraxanthine or 1-methylxanthine alone.

### Weight Loss

According to certain embodiments, fat loss is promoted through inducing thermogenesis in the SCM subject. According to exemplary implementations of these embodiments, the composition may also include one or more compounds selected from: caffeine, green tea, capsaicin, garcinia cambogia, yohimbine, catechols, epigallocatechin gallate EGCG, catechins, and proanthocyanidins and octacosanol, Synephrine, theacrine, methylliberine, cayenne, grains of paradise, ginger extract, and bitter orange.

According to further embodiments, fat loss is promoted through suppression of appetite in the subject. In exemplary implementations of these embodiments, the composition further may include one or more compounds selected from: fenugreek, glucomannan, gymnema sylvestre, 5-HTP, Caralluma fimbriata, green tea extract, conjugated linoleic acid, Garcinia cambogia, and Yerba mate.

According to still further embodiments, fat loss is promoted through enhancing lipolysis in the SCM subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides, BAIBA, grains of paradise, ginger and octacosanol.

In certain embodiments, the disclosed composition, when administered to a subject, increases the subjects resting energy expenditure, relative to the subject’s baseline level or following administration of a placebo. In certain embodiments, the increase in the subject’s resting energy expenditure following administration of the disclosed compositions is from about 3% to about 30%. In further embodiments, increase in the subject’s resting energy expenditure following administration of the disclosed compositions is from about 5% to about 25%. In yet further embodiments increase in the subject’s resting energy expenditure following administration of the disclosed compositions is from about 8% to about 20%. In still further embodiments, increase in the subject’s resting energy expenditure following administration of the disclosed compositions is from about 10%.

According to certain implementations, the disclosed method further comprises restricting calorie intake of the SCM subject. In exemplary implementations, the amount of fat loss in the subject is greater than that for a subject with an equivalent calorie restriction that has not been provided the composition. According to further implementations, the ratio of fat loss to muscle loss in the subject the subject is greater than that for a subject with an equivalent calorie restriction that has not been provided the composition.

Further disclosed herein are methods for suppressing appetite in a SCM subject by administering to the subject a composition comprising about 2 mg to about 800 mg of paraxanthine. In certain embodiments, administration of the composition to the subject reduces the subject’s appetite by from 5% to about 70%. In further embodiments, reduction of the subject’s appetite is from about 10% to about 60%. In yet further embodiments, reduction of the subject’s appetite is from about 20% to about 50%. In still further embodiments, reduction of the subject’s appetite is at least about 30%.

According to certain embodiments of the disclosed method, the composition is administered in a therapeutically effective amount. In further embodiments, the composition is administered in a prophylactically effective amount.

In certain aspects, disclosed herein are methods to promote weight loss in a SCM subject through the administration of an effective amount of one or more compositions disclosed herein. According to certain aspects, administration of effective amounts of the disclosed compositions are used in treating diabetes mellitus; preventing, slowing progression of, delaying or treating of a condition or disorder selected from the group consisting of complications of diabetes mellitus; preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity, metabolic syndrome and gestational diabetes; or improving glycemic control and/or for reducing of fasting plasma glucose, of postprandial plasma glucose and/or of glycosylated hemoglobin HbA1c; or preventing, slowing, delaying or reversing progression from impaired glucose tolerance IGT), impaired fasting blood glucose (IFG), insulin resistance and/or from metabolic syndrome to type 2 diabetes mellitus; or preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, arteriosclerosis, myocardial infarction, stroke and peripheral arterial occlusive disease; or reducing body weight and/or body fat or preventing an increase in body weight and/or body fat or facilitating a reduction in body weight and/or body fat; or preventing, slowing, delaying or treating diseases or conditions attributed to an abnormal accumulation of ectopic fat; or maintaining and/or improving the insulin sensitivity and/or for treating or preventing hyperinsulinemia and/or insulin resistance; preventing, slowing progression of, delaying, or treating new onset diabetes after transplantation (NODAT) and/or post-transplant metabolic syndrome (PTMS); preventing, delaying, or reducing NODAT and/or PTMS associated complications including micro- and macrovascular diseases and events, graft rejection, infection, and death; treating diabetes associated with cystic fibrosis treating hyperuricemia and hyperuricemia associated conditions; treating or prevention kidney stones; treating hyponatremia; in a SCM subject in need thereof.

Another aspect encompasses a combination therapy to regulate fat storage, energy utilization, and/or weight loss in a SCM subject. In an exemplary embodiment, a combination for increasing energy utilization, decreasing body fat or for promoting weight loss may include combining the methods and compositions disclosed with a procedure or therapy such as a pharmaceutical therapy, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, brown adipose tissue modulation (e.g., controlled activation, enhanced differentiation, supplemental implantation, etc.), cryolipolysis, pharmaceutical administration, electrical stimulation of nerves that innervate at least a portion of the gastrointestinal tract, therapies impacting circadian rhythms, bile acid modulation, intestinal mucus production and metabolism, duodenal endoluminal barrier or similar manipulations of the gastrointestinal tract. For example, a composition dileucine can be administered to the SCM subject prior to, concurrently with or after a gastric bypass or other gastrointestinal or bariatric procedure.

In certain aspects, administration of the disclosed compositions in a SCM subject is effective at preventing reducing body weight and/or body fat or preventing an increase in body weight and/or body fat or facilitating a reduction in body weight and/or body fat; or preventing, slowing, delaying or treating diseases or conditions attributed to an abnormal accumulation of liver fat; or maintaining and/or improving the insulin sensitivity and/or for treating or preventing hyperinsulinemia and/or insulin resistance.

### Cognitive Function

Disclosed herein is a method of enhancing cognitive function in a SCM subject comprising administering to the subject a composition disclosed herein. In certain embodiments, improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

In certain embodiments, administration of the disclosed composition increases working memory in a SCM subject.

In further embodiments, administration of the disclosed composition increases attention in a SCM subject.

Brain Derived Neurotrophic Factor (BDNF) is a key molecule involved in plastic changes related to learning and memory (Miranda et al. Brain-Derived Neurotrophic Factor: A Key Molecule for Memory in the Healthy and the Pathological. Brain. Front. Cell. Neurosci. 2019, 13:363.). Increases in BDNF are linked to improved cognition. According to certain embodiments, administration of disclosed compositions to SCM subjects results in increased plasma BNDF levels in the subject. In certain implementations, administration of the disclosed composition increases serum BDNF concentration from about 30-60% relative to SCM subjects who received placebo. In further embodiments, administration of the disclosed composition increases serum BDNF concentration about 50% relative to SCM subjects who received placebo. In yet further embodiments, administration of the disclosed composition increases serum BDNF concentration of at least about 10% relative to fast caffeine metabolizers who received a comparable dose of the composition. In yet further embodiments, administration of the disclosed composition increases serum BDNF concentration about 50% relative to SCM subjects who received caffeine.

According to certain embodiments, composition of the instantly disclosed methods to enhance cognitive function in a SCM subject further comprise tyrosine, N-acetyl-tyrosine, taurine, huperzine A, acetyl-1-carnitine, CDP choline, Alpha GPC, choline bitrate, choline citrate, B12, caffeine, methyllliberine, theacrine, paraxanthine, theobromine, ashwagandha, rhodiola, lutein, zeaxanthin, fish oil, creatine, ginseng, lions mane, niacin, cordyceps, theanine, B-vitamins, GABA, sulbutiamine, vinpocetine, adenosine triphosphate, inositol, enhanced arginine silicate, nitrates, electrolytes, hesperidin and derivatives of hesperidin and/or bacopa.

### Methods of Treatment

Further disclosed herein is a method of treating a condition in a SCM subject in need thereof by administering to the SCM subject a composition disclosed herein. In certain embodiments, the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson’s disease, Alzheimer’s, and dementia.

Further disclosed herein is a method for treating a mood disorder by administering to a subject in need thereof a composition disclosed herein. In certain embodiments, the mood disorder is selected from clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, comorbid depression, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein.

In certain embodiments, the mood disorder is depression. In exemplary implementations, subject has been diagnosed with depression or is at risk of depression.

Further disclosed herein is a method for treating an anxiety disorder in a subject in need thereof by administering to a subject in need thereof a composition disclosed herein. In certain embodiments, the anxiety disorder is selected from: generalized anxiety disorder, panic disorder, obsessive compulsive disorder, phobia, post-traumatic stress disorder). As will be appreciated by those skilled in the art, anxiety disorder is a blanket term covering several different forms of abnormal and pathological fear and anxiety.

According to certain embodiments, the composition is administered in a therapeutically effective amount. In further embodiments, the composition is administered in a prophylactically effective amount.

In certain embodiments, the composition used in the method of treating a mood disorder or anxiety disorder further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, taurine, tyrosine, n-acetyl-tyrsoine, macuna, sceletium tortuosa, 5-HTP, tryptophan, saffron, Vitamin D, SAMe, lions mane and/or huperzine A.

Further disclosed herein is a method for treating or preventing age-related cognitive decline in a SCM subject in need thereof, comprising administering to the subject an effective amount of a composition disclosed herein. In certain embodiments, administration of the composition increases one or more of attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition. In certain implementations, administration of the composition to the subject increases levels of catalase and/or glutathione in the subject. In further implementations, the composition administered to the subject comprises paraxanthine and 1-methylxanthine and the administration of paraxanthine and 1-methylxanthine produce a synergistic increase in catalase and/or glutathione in the subject, relative to the administration of paraxanthine or 1-methylxanthine alone.

Further disclosed herein is a method for treating or preventing Alzheimer’s disease in SCM subject in need thereof, comprising administering to the subject an effective amount of a composition disclosed herein. In certain implementations, administration of the composition to the subject decreases the level of amyloid β-protein (Aβ) in the subject. In certain embodiments, subject has been diagnosed with Alzheimer’s disease. In further embodiments, the subject is at risk of Alzheimer’s disease. In yet further embodiments, the subject has been diagnosed with mild cognitive impairment.

In certain aspects, the disclosed compositions are a neuroprotective agent. In certain embodiments, administration of the disclosed compositions to a subject in need thereof is neuroprotective. In exemplary aspects of these embodiments, this neuroprotection is in the form of protecting against dopaminergic cell death.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The administration of the disclosed compositions to a SCM subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit-other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present invention are defined by the following numbered clauses:
1. A composition comprising a first active ingredient comprising about from 2 mg to about 800 mg paraxanthine.
2. The composition of clause 1, wherein paraxanthine is present in amount from about 20 mg to about 600 mg.
3. The composition of clause 2, wherein paraxanthine is present in amount from 50 mg to about 400 mg.
4. The composition of clauses 1-3, further comprising a second active ingredient, selected from a group consisting of: L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, gallic acid, (+)-catechin (C), (-)-epicatechin (EC), (+)-gallocatechin (GC), (-)-epigallocatechin (EGC), (-)-catechin gallate (CG), (-)-gallocatechin gallate (GCG), (-)-epicatechin gallate (ECG) and (-)-epigallocatechin gallate (EGCG), glycerides, propylene glycol, lauroyl macrogol, lauroyl macrogol derivatives, co-crystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger &gingerols/shogaols), hoodia gordonii, caffeine, yohimbine, methylsynephrine, synephrine, theobromine, tocopherols, theophylline, alpha-yohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, ginkgo biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, medium chain triglycerides, creatine, citrulline, arginine, lions mane, cordyceps, leucine, isoleucine, valine, BAIBA, ergothioneine, grains of paradise, Kanna, Huperzine A, ketones, Maca, ginseng, ashwagandha, rhodiola, theanine BCAAs, beta-alanine, fish oil, citrulline, arginine, HMB, HICA, balenine, carnosine, anserine and combinations thereof.
5. The composition of clause 1, further comprising a paraxanthine congener or paraxanthine analog.
6. The composition of clause 5, wherein said paraxanthine congener or analog is selected from the group consisting of caffeine, 7-methylxanthine, 3-methylxanthine, 1-methylxanthine, theobromine, theophylline, liberine, methylliberine, and combinations thereof.
7. The composition of clause 6, wherein the paraxanthine congener or analog is caffeine.
8. The composition of clause 7, wherein the effective dose of caffeine is lower than the effective dose of caffeine in a composition without paraxanthine.
9. A composition comprising paraxanthine and 1-methylxanthine.
10. The composition of clause 9, wherein the paraxanthine and 1-methylxanthine are each present in an amount from about 2 mg to about 800 mg.
11. The composition of clause 10, wherein the paraxanthine and 1-methylxanthine are each present in an amount from about 20 mg to about 600 mg.
12. The composition of clause 11, wherein the paraxanthine and 1-methylxanthine are each present in an amount from about amount from 50 mg to about 400 mg.
13. The composition of any of clauses 9-12, further comprising one or more of an active selected from a group consisting of: L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, gallic acid, (+)-catechin (C), (-)-epicatechin (EC), (+)-gallocatechin (GC), (-)-epigallocatechin (EGC), (-)-catechin gallate (CG), (-)-gallocatechin gallate (GCG), (-)-epicatechin gallate (ECG) and (-)-epigallocatechin gallate (EGCG), glycerides, propylene glycol, lauroyl macrogol, lauroyl macrogol derivatives, co-crystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alpha-yohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, ginkgo biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, medium chain triglycerides, creatine, citrulline, arginine, lions mane, cordyceps, leucine, isoleucine, valine, BAIBA, ergothioneine, grains of paradise, Kanna, Huperzine A, ketones, Maca, ginseng, ashwagandha, rhodiola, theanine BCAAs, beta-alanine, fish oil, citrulline, arginine, HMB, HICA, balenine, carnosine, anserine and combinations thereof.
14. A composition comprising 1-methylxanthine and 7-methylxanthine.
15. The composition of clause 14, wherein the 1-methylxanthine and 7-methylxanthine are each present in an amount from about 2 mg to about 800 mg.
16. The composition of clause 15, wherein the 1-methylxanthine and 7-methylxanthine are each present in an amount from about 20 mg to about 600 mg.
17. The composition of clause 16, wherein the 1-methylxanthine and 7-methylxanthine are each present in an amount from about amount from 50 mg to about 400 mg.
18. The composition of any of clauses 14 to 17, further comprising one or more active ingredient, selected from a group consisting of: L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, gallic acid, (+)-catechin (C), (-)-epicatechin (EC), (+)-gallocatechin (GC), (-)-epigallocatechin (EGC), (-)-catechin gallate (CG), (-)-gallocatechin gallate (GCG), (-)-epicatechin gallate (ECG) and (-)-epigallocatechin gallate (EGCG), glycerides, propylene glycol, lauroyl macrogol, lauroyl macrogol derivatives, co-crystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, tocopherols, theophylline, alpha-yohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline,synephrine, yohimbine, rhodiola, ashwagandha, ginseng, ginkgo biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, medium chain triglycerides, creatine, citrulline, arginine, lions mane, cordyceps, leucine, isoleucine, valine, BAIBA, ergothioneine, grains of paradise, Kanna, Huperzine A, ketones, Maca, ginseng, ashwagandha, rhodiola, theanine BCAAs, beta-alanine, fish oil, citrulline, arginine, hydroxy-methyl-butyrate, HICA, balenine, carnosine, anserine, carbonate, probiotic, and combinations thereof.
19. The composition of any preceding clause, wherein the is a powder.
20. The composition of any preceding clause, wherein the supplement is in a solid oral dosage form.
21. The composition of any preceding clause, wherein the supplement is formulated for topical administration.
22. The composition of any preceding clause, except clauses 7-8, wherein the composition is substantially free of caffeine.
23. A method for improving energy in a slow caffeine metabolizer (SCM) subject, comprising:
   a. identifying an individual as a SCM subject; and
   b. providing the SCM subject with the composition of any of claims 1-23.
24. The method of clause 23, wherein upon administration of the composition, the subject experiences improvement of at least one of mood, energy, focus, concentration or sexual desire or a reduction of at least one of anxiety, fatigue, perception of effort or perception of pain.
25. The method of clause 24, wherein upon continued administration to the subject, the composition does not create dependence in the subject and/or withdrawal effect in the subject when continued use is ceased.
26. The method of clause 23, wherein the amount of paraxanthine administered is from about 50 mg to about 400 mg.
27. The method of clause 23, wherein the subject experiences a decrease in fatigue of at least about 6 percent.
28. The method of clause 23, wherein the SCM subject experiences an increase in energy of at least about 5 percent.
29. The method of clause 23, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, taurine, tyrosine, n-acetyl-tyrsoine, macuna, sceletium tortuosa, 5-HTP, tryptophan, saffron, Vitamin D, SAMe, lions mane and huperzine A.
30. A method of increasing athletic endurance in a SCM subject comprising administering to the subject the composition of any of clauses 1-13.
31. The method of clause 30, wherein the composition is the composition of any of clauses 9-13, and wherein the administration of paraxanthine and 1-methylxanthine produce a synergistic increase athletic endurance in the subject, relative to the administration of paraxanthine or 1-methylxanthine alone.
32. A method of treating a condition in a SCM subject in need thereof, comprising administering to the subject the composition of any of clauses 1-13.
33. The method of clause 32, wherein the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), concussion, Parkinson’s disease, Alzheimer’s, and dementia.
34. The method of clause 32, wherein the condition is a mood disorder.
35. The method of clause 34, wherein the mood disorder is depression.
36. The method of clause 35, wherein the subject has been diagnosed with depression or is at risk of depression.
37. The method of clause 33, wherein the condition is an anxiety disorder.
38. The method of clause 33, wherein the composition is administered in a therapeutically effective amount.
39. The method of clause 33, wherein the composition is administered in a prophylactically effective amount.
40. The method of clause 33, wherein the composition comprises 1-paraxanthine at an amount from about 2 mg to about 800 mg.
41. The method of clause 32, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino) ethanol (DMAE), DMAE bitartrate, magnolia bark, theanine, phosphatidylserine, ashwagandha, rhodiola, taurine, tyrosine, n-acetyl-tyrsoine, macuna, sceletium tortuosa, 5-HTP, tryptophan, saffron, Vitamin D, SAMe, lions mane and/or huperzine A.
42. A method of enhancing attention in a SCM subject in need thereof comprising administering the composition of any of clauses 1-22.
43. A method of improving working memory in a subject in need thereof comprising administering a composition to the subject comprising the composition of any of clauses 1-22.
44. A method of improving cognitive performance in a SCM subject comprising administering the composition of any of clause 1-22.
45. The method of clause 44, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.
46. The method of clause 44 - 45, wherein the subject has experience age-related cognitive decline.
47. The method clauses 44-46, wherein administration of the composition to the subject increases the level BDNF in the subject.
48. A method for treating or preventing age-related cognitive decline in a SCM subject in need thereof, comprising administering to the subject an effective amount of the composition of any of clauses 1-22.
49. The method of clause 48, wherein administration of the composition increases one or more of attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.
50. The method of clause 49, wherein administration of the composition to the subject increases levels of catalase and/or glutathione in the subject.
51. The method of clause 50, wherein the composition is the composition of any of clauses 9-13, and wherein the administration of paraxanthine and 1-methylxanthine produce a synergistic increase in catalase and/or glutathione in the subject, relative to the administration of paraxanthine or 1-methylxanthine alone.
52. The method of clause 49, wherein administration of the composition to the subject increases BDNF in the subject.
53. The method of clause 51, wherein the composition is the composition of any of clauses 9-13, and wherein the administration of paraxanthine and 1-methylxanthine produce a synergistic increase in BDNF in the subject, relative to the administration of paraxanthine or 1-methylxanthine alone.
54. The method of clause 49, wherein administration of the composition to the subject decreases the level of amyloid P-protein (Aβ) in the subject.
55. The method of clause 54, wherein administration of the composition is the composition of any of clauses 9-13, and wherein the administration of paraxanthine and 1-methylxanthine produce a synergistic decrease in Aβ in the subject, relative to the administration of paraxanthine or 1-methylxanthine alone.
56. A method for treating or preventing Alzheimer’s disease in a SCM subject in need thereof, comprising administering to the subject an effective amount of the composition of any of clauses 1-22.
57. The method of clause 56, wherein administration of the composition is the composition of any of clauses 9-13, and wherein the administration of paraxanthine and 1-methylxanthine produce a synergistic decrease in Aβ in the subject, relative to the administration of paraxanthine or 1-methylxanthine alone.
58. The method of clause 56, wherein the subject has been diagnosed with Alzheimer’s disease.
59. The method of clause 56, wherein the subject is at risk of Alzheimer’s disease.
60. The method of clause 56, wherein the subject has been diagnosed with mild cognitive impairment.
61. A nutritional supplement for improving muscle strength, muscle size, and/or muscle function comprising the composition of any of clauses 1-22.
62. The nutritional supplement of clause 69, wherein the nutritional supplement is powder or a capsule.
63. A method of increasing muscle size in a subject comprising administering to the subject in need thereof with an effective amount of the composition of any of clauses 1-22.
64. The method of clause 74, wherein the composition is the composition of any of clauses 9-13, and wherein the administration of paraxanthine and 1-methylxanthine produce a synergistic increase in muscle size in the subject, relative to the administration of paraxanthine or 1-methylxanthine alone.
65. A method for promoting weight loss in a SCM subject, comprising: administering to the subject the composition of any of clauses 1-22.
66. The method of clause 76, wherein weight loss is promoted through inducing thermogenesis in the subject.
67. The method of clause 77, wherein the composition further comprises one or more compounds selected from a list consisting of: caffeine, green tea, capsaicin, garcinia cambogia, yohimbine and bitter orange.
68. The method of clause 76, wherein weight loss is promoted through suppression of appetite in the subject and wherein administration of the composition to the subject suppresses appetite in thesubject by at least about 30%.
69. The method of clause 76, wherein weight loss is promoted through enhancing lipolysis in the subject.
70. The method of clause 76, wherein administration of the composition to the subject decreases the respiratory quotient in the subject by at least about 10%.
71. The method of clause 76, wherein resting energy expenditure in the subject is increased by at least about 15%.
72. The method of clause 80, wherein the wherein the composition further comprises one or more compounds selected from a list consisting of caffeine, green tea extract, L-carnitine, Garcinia cambogia (hydroxycitric acid), capsaicin, ginseng, taurine, silk peptides, catechols, epigallocatechin gallate (EGCG), catechins, proanthocyanidins and octacosanol.
73. The method of clause 76, further comprising restricting calorie intake of the subject and wherein the amount of weight loss in the subject is greater than that for a subject with an equivalent calorie restriction that has not been provided the composition and wherein the ratio of fat loss to muscle loss in the subject is greater than that for a subject with an equivalent calorie restriction that has not been provided the composition.
74. The method of clause 76, wherein the subject is not administered caffeine.
75. A method for suppressing appetite in a SCM subject comprising: administering to the subject the composition of any of clauses 1-22.
76. The method of clause 86, wherein administration of the composition decreases appetite in the subject by at least about 30%.
77. The method of clause 86, wherein the subject is not administered caffeine.
78. A method for promoting fat loss in a SCM subject, comprising: administering to the subject with the composition of any of clauses 1-13.
79. The method of clauses 89, wherein the subject is not administered caffeine.
80. The method of clause 89, wherein fat loss is promoted through inducing thermogenesis in the subject.
81. The method of clause 91, wherein the composition further comprises one or more compounds selected from the list consisting of: caffeine, green tea, capsaicin, garcinia cambogia, yohimbine, catechols, EGCG, catechins, and proanthocyanidins and octacosanol and bitter orange.
82. The method of clause 89, wherein fat loss is promoted through suppression of appetite in the subject.
83. The method of clause 93, wherein administration of the composition to the subject suppresses appetite in the subject by at least about 30%.
84. The method of clause 89, wherein administration of the composition to the subject decreases the respiratory quotient in the subject by at least about 10%.
85. The method of clause 93, wherein the composition further comprises one or more compounds selected from the list consisting of: fenugreek, glucomannan, gymnema sylvestre, 5-HTP, Caralluma fimbriata, green tea extract, conjugated linoleic acid, Garcinia cambogia, and Yerbamate.
86. The method of clause 89, wherein fat loss is promoted through enhancing lipolysis in the subject.
87. The method of clause 97, wherein the wherein the composition further comprises one or more compounds selected from the list consisting of caffeine, green tea extract, L-carnitine, Garcinia cambogia (hydroxycitric acid), capsaicin, ginseng, taurine, silk peptides and octacosanol.
88. The method of clause 89, further comprising restricting calorie intake of the subject.
89. The method of clause 99, wherein the amount of fat loss in the subject is greater than that for a subject with an equivalent calorie restriction that has not been provided the composition.
90. The method of clause 99, wherein the ratio of fat loss to muscle loss in the is greater than that for a subject with an equivalent calorie restriction that has not been provided the composition.
91. A composition for increasing energy in a SCM subject comprising 1-methylxanthine and paraxanthine.
92. The composition of clause 102, wherein the paraxanthine and 1-methylxanthine are each present in an amount from about 2 mg to about 800 mg.
93. The composition of clause 103, wherein the paraxanthine and 1-methylxanthine are each present in an amount from about amount from 50 mg to about 400 mg.
94. The composition of clause 102, further comprising an active agent, selected from a group consisting of: L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, gallic acid, (+)-catechin (C), (-)-epicatechin (EC), (+)-gallocatechin (GC), (-)-epigallocatechin (EGC), (-)-catechin gallate (CG), (-)- gallocatechin gallate (GCG), (-)-epicatechin gallate (ECG) and (-)-epigallocatechin gallate (EGCG), glycerides, propylene glycol, lauroyl macrogol, lauroyl macrogol derivatives, co-crystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger &gingerols/shogaols), hoodia gordonii, caffeine, yohimbine, methylsynephrine, synephrine, theobromine, tocopherols, theophylline, alpha-yohimbine, conjugated linoleic acid (CLA), octopamine, evodiamine, passion flower, red pepper, cayenne, raspberry ketone, guggul, green tea, guarana, kola nut, beta- Phenethylamines, Acacia rigidula, forskolin (Coleus forskohlli), theophylline, synephrine, yohimbine, rhodiola, ashwagandha, ginseng, ginkgo biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5- hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, forskolin (Coleus forskohlli), 2-(dimethylamino)ethanol (DMAE), DMAE bitartrate, medium chain triglycerides, creatine, citrulline, arginine, lions mane, cordyceps, leucine, isoleucine, valine, BAIBA, ergothioneine, grains of paradise, Kanna, Huperzine A, ketones, Maca, ginseng, ashwagandha, rhodiola, theanine BCAAs, beta-alanine, fish oil, citrulline, arginine, hydroxy-methylbuterate, HICA, balenine, carnosine, anserine and combinations thereof.
95. The composition of clause 102, wherein administration of the composition to a subject produces a synergistic increase in energy relative to the administration of a comparable dose of paraxanthine or 1-methylxanthine alone.
96. The composition of clause 102, wherein 1-methylxanthine and paraxanthine are present at a ratio of about 4:1 to about 1:4.
97. A method for increasing energy in a SCM subject comprising administering to the subject a composition comprising an effective amount of 1-methylxanthine.
98. The method of clause 108, wherein the amount of 1-methylxanthine administered is from about 2 mg to about 800 mg.
99. The method of clause 108, wherein the subject experiences and increase in perception of energy of at least about 5%.
100. The method of clause 108, wherein the subject experiences a decrease of at least one of anxiety, fatigue, perception of effort, and/or perception of pain.
101. The method of clause 108, wherein the composition further comprises paraxanthine in an amount from about 2 mg to about 800 mg.
102. The method of clause 112, wherein the administration of paraxanthine and 1-methylxanthine produce a synergistic increase in perception of energy in the subject, relative to the administration of a comparable dose of paraxanthine or 1-methylxanthine alone.
103. The method of clause 108, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, taurine, tyrosine, n-acetyl-tyrsoine, macuna, sceletium tortuosa, 5-HTP, tryptophan, saffron, Vitamin D, SAMe, lions mane and huperzine A.
104. The method of clause 108, wherein the composition is substantially free of caffeine.
105. A method for improving athletic performance in a SCM subject in comprising administering to the subject a composition comprising an effective amount of 1-methylxanthine.
106. The method of clause 115, wherein the amount of 1-methylxanthine administered is from about 50 mg to about 400 mg.
107. The method of clause 116, wherein athletic performance is increased by at least about 10 %.
108. The method of clause 116, wherein the subject experiences and increase in endurance.
109. The method of clause 116, wherein the composition further comprises paraxanthine in an amount from about 2 mg to about 800 mg and wherein administration of the composition to a subject produces a synergistic increase in athletic performance to the administration of a comparable dose of paraxanthine or 1-methylxanthine alone.
110. The method of clause 116, wherein the composition further comprises at least one agent selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, huperzine A, theacrine, methylliberine, B12, sulbutiamine, magnolia bark, ketones, MCTs, omega 3's, lutein, zeaxanthin, tyrosine and n-acetyl-tyrosine, taurine, acetyl-1-carnitine and/or combinations thereof.
111. The method of any of clauses 23-110, further comprising the step of identifying the SCM subject by genotyping the individual for a genetic variant associated with slow caffeine metabolism.
112. The method of clause 111, wherein the genetic variant is in a CYP1A2, ADORA2A, and/or CFP2E1 gene.
113. The method of claim 112, wherein the individual is identified as a SCM subject if the individual carries a CYP1A2*1F variant.
114. The method of any of clauses 23-113, further comprising the step of identifying the SCM subject by administering the individual a questionnaire on the individual’s response to caffeine.
115. 114. The method of any of clauses 23-113, further comprising the step of identifying the SCM subject by measuring the individual’s rate of caffeine metabolism.
116. A caffeine substitute comprising from about 2 mg to about 800 mg paraxanthine, wherein the caffeine substitute has reduced bitterness relative to a comparable amount of caffeine.
117. A decaffeinated beverage, comprising the caffeine substitute of clause 116.
118. The decaffeinated beverage of clause 117, wherein the decaffeinated product is a coffee product.
119. The decaffeinated beverage of clause 117, wherein the decaffeinated product is a tea product.
120. The decaffeinated beverage of clause 117, wherein the decaffeinated product is a soft drink or energy drink.
121. The caffeine substitute of clause 116, further comprising a paraxanthine congener or paraxanthine analog.
122. The caffeine substitute of clause 121, wherein said paraxanthine congener or analog is selected from the group consisting of caffeine, 1-methylxanthine, 7-methylxanthine, theobromine, theophylline, liberine, methylliberine, and combinations thereof.
123. The caffeine substitute of clause 122, wherein the paraxanthine congener or analog is caffeine.
124. The caffeine substitute of clause 123, wherein the effective dose of caffeine is lower than the effective dose of caffeine in a composition without paraxanthine.
125. The caffeine substitute of any of clauses 116-122, wherein the caffeine substitute is substantially free of caffeine.
126. A method for improving physical performance or energy in subject, comprising: providing the subject with a caffeine substitute comprising about 2 mg to about 800 mg of paraxanthine, either natural or synthetic.
127. The method of clause 126, wherein upon administration of the caffeine substitute, the subject experiences improvement of at least one of mood, energy, focus, concentration or sexual desire or a reduction of at least one of anxiety, fatigue, perception of effort or perception of pain.
128. The method of clause 127, wherein upon continued administration to the subject, the caffeine substitute does not create dependence in the subject and/or withdrawal effect in the subject when continued use is ceased.
129. The method of clause 126, wherein the amount of paraxanthine about 50 mg to about 400 mg.
130. The method of clause 126, wherein the subject experiences a decrease in fatigue of at least about 6 percent.
131. The method of clause 126, wherein the subject experiences an increase in energy of at least about 5 percent.
132. The method of clause 126, wherein the caffeine substitute further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, paraxanthine, 1-methylxanthine, 7-methylxanthine, theobromine, naringin, hesperidin, 2-(dimethylamino)ethanol (DMAE), DMAE bitartrate, huperzine A, nicotinamide mononucleotide (NMN), exogenous ketones, medium chain triglyceride (MCTs), Ergothioneine, berberine, dihydroberberine and combinations thereof.
133. A method of enhancing attention in a subject in need thereof comprising administering a caffeine substitute to the subject comprising paraxanthine.
134. A method of improving working memory in a subject in need thereof comprising administering a caffeine substitute to the subject comprising paraxanthine.
135. A method of improving cognitive performance in a subject comprising administering a caffeine substitute to the subject comprising paraxanthine.
136. A method of aiding weight loss and/or fat loss in a subject comprising administering a caffeine substitute to the subject comprising paraxanthine.
137. The method of clause 136, wherein the weight loss results from increased metabolism in the subject.
138. The method of clause 136, wherein the weight loss results from decreased caloric intake in the subject.
139. A caffeine substitute composition for use in a decaffeinated product comprising paraxanthine.
140. The composition of clause 139, wherein the decaffeinated product is perceived as less bitter when consumed than a comparable caffeinated product.
141. The composition of clause 139, wherein the composition does not increase anxiety when administered to a subject relative to a comparable dose of caffeine.
142. The composition of clause 139, wherein the composition does not create dependence in a subject upon repeated administrations and does not create withdrawal effects in the subject upon cessation of use.
143. The composition of clause 139, where the composition is less bitter than a comparable dose of caffeine.
144. The composition of clause 139, where the composition is less toxic than a comparable dose of caffeine.
145. A method of preparing a decaffeinated product retaining the benefits of caffeine comprising applying paraxanthine to the decaffeinated product in an amount substantially similar to the amount of caffeine in a comparable caffeinate product.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### EXAMPLE 1 N-Methyl Nicotinamide (Energy, Weight Management, Fat Loss)

### Methods

### Study Design

### 1. Design

Randomized, double-blind, cross-over, four arm, four treatments, two period, controlled study.

### 2. Number of Subjects

24 subjects with 6 subjects in each of the 4 arms in Period I. After a washout period of 2-weeks, same subjects participated again in Period II. A total of 24 subject’s data were analyzed towards the end of the study.

Table 2

Baseline characteristics

| Parameter/Statistics | Overall |
|---|---|
| Age (Years) | |
| N | 24 |
| Mean (SD) | 30.2 (7.31) |
| Median | 27.5 |
| Min, Max | 19,46 |
| Sex, n (%) | |
| Female | 11 (45.8) |
| Male | 13 (54.2) |
| Medical History, n (%) | |
| Yes | 0 (0.0) |
| No | 24 (100.0) |
| Concomitant Medication, n (%) | |
| Yes | 0 (0.0) |
| No | 24 (100.0) |
| Normal | 24 (100.0) |
| Abnormal | 0 (0.0) |

### 3. Randomization

Investigational products duly labeled with randomization codes were provided to the investigators by the sponsor. Per the randomization schedule the investigator / designee were dispense IPs on Day 0. The IPs was kept by the investigator in a safe but accessible place.

### 4. Supplementation

### Period I

Arm 1: Decaffeinated beverage plus 50 mg Caffeine (3 slow and 3 fast metabolizers)

Arm 2: Decaffeinated beverage plus 200 mg Caffeine (3 slow and 3 fast metabolizers)

Arm 3: Decaffeinated beverage plus 50 mg enfinityⓇ Paraxanthine (3 slow and 3 fast metabolizers)

Arm 4: Decaffeinated beverage plus 200 mg enfinityⓇ Paraxanthine (3 slow and 3 fast metabolizers)

Post completion of seven days treatment in Period I, subjects were asked to visit the site after 14 days of washout period. The same set of subjects participated in Period II. They were administered the following doses of Investigational products;

### Period II

Arm 1: Decaffeinated beverage plus 50 mg Placebo (same group received 50 mg of caffeine in Period I) - same 6 subjects of Period I

Arm 2: Decaffeinated beverage plus 200 mg Placebo (same group received 200 mg of caffeine in Period I) - same 6 subjects of Period I

Arm 3: Decaffeinated beverage plus 50 mg Placebo (same group received 50 mg of Paraxanthine in Period I) - same 6 subjects of Period I

Arm 4: Decaffeinated beverage plus 200 mg Placebo (same group received 200 mg of Paraxanthine in Period I) - same 6 subjects of Period I

Each group of 6 subjects had a subset of 3 slow and 3 fast caffeine metabolizers. In total, all the 4 groups/arms had 12 slow caffeine metabolizers and 12 fast caffeine metabolizers. A total of 24 subject’s data was analyzed towards the end of the study.

### 5. Genomic Testing

The blood samples were collected, and the genomic sequencing was analyzed, on the Basis of reports the subjects were divided into 2 groups having slower metabolism & faster metabolism.

### 6. Blood sample

3 ml of blood was withdrawn in each visit from each subject and N-methylnicotinamide (MNA) as a biomarker of energy was analyzed by LCMS. MNA is a novel myokine that enhances the utilization of energy stores in response to low muscle energy availability (Ström et al. N1-methylnicotinamide is a signaling molecule produced in skeletal muscle coordinating energy metabolism. Sci Rep 2018, 8:3016).

### 7. Results

Paraxanthine ingestion for 7 days showed dose-dependent greater increases in MNA compared to placebo, and compared to caffeine. Subgroup analysis of the overall results by fast and slow metabolizers of caffeine showed that paraxanthine ingestion resulted in significantly greater increases in MNA in slow metabolizers of caffeine when compared to caffeine ingestion in slow metabolizers of caffeine.

### 50 mg Paraxanthine (n=6) vs. 50 mg Placebo (n=6)

50 mg paraxanthine (n=6) significantly increased MNA by 21.7% from 9.35±3.10 ng/ml to 11.38±1.85 ng/ml compared to placebo (p<0.05). MNA decreased in the placebo group (n=6) by 21.6%, from 7.41±2.13 ng/ml to 5.81±5.73 ng/ml.

### 50 mg Caffeine (n=6) vs. 50 mg Placebo (n=6)

50 mg caffeine (n=6) non-significantly increased MNA by 13.6% from 8.89±3.08 ng/ml to 10.10±2.06 ng/ml compared to placebo (p=0.1147). MNA decreased in the placebo group (n=6) by 28.9%, from 7.78±1.49 ng/ml to 5.53±3.89 ng/ml.

### 50 mg Paraxanthine (n=6) vs. 50 mg Caffeine (n=6)

50 mg paraxanthine showed a 66.8% greater increase in MNA than 50 mg caffeine. 200 mg paraxanthine (n=6) vs. 200 mg placebo (n=6)

200 mg paraxanthine (n=6) significantly increased MNA by 40.0% from 8.91±1.45 ng/ml to 12.47±1.61 ng/ml compared to placebo (p<0.05). MNA decreased in the placebo group (n=6) by 20.1%, from 5.68±2.81 ng/ml to 4.54±2.58 ng/ml.

### 200 mg Caffeine (n=6) vs. 200 mg Placebo (n=6)

200 mg caffeine (n=6) non-significantly increased MNA by 22.2 % from 8.54±2.60 ng/ml to 10.44±1.24 ng/ml compared to placebo (p=0.4335). MNA increased in the placebo group (n=6) by 3.7 %, from 5.85±3.65 ng/ml to 6.07±3.06 ng/ml.

### 200 mg Paraxanthine (n=6) vs. 200 mg Caffeine (n=6)

200 mg paraxanthine showed an 86.9% greater increase in MNA than 200 mg caffeine.

### 50 mg Paraxanthine (Slow Metabolizer, n=3) vs. 50 mg Placebo (Slow Metabolizer, n=3)

Ingestion of 50 mg paraxanthine in slow metabolizer of caffeine (n=3) for 7 days significantly increased MNA by 48.3% from 8.36±2.20 ng/ml to 12.40±1.03 ng/ml. MNA increased in the placebo group (n=3, slow metabolizer of caffeine) by 12.6%, from 7.67±1.73 ng/ml to 8.64±5.90 ng/ml.

### 50 mg Caffeine (Slow Metabolizer, n=3) vs. 50 mg Placebo (Slow Metabolizer, n=3)

Ingestion of 50 mg caffeine in slow metabolizer of caffeine (n=3) for 7 days increased MNA by 22.2% from 8.73±1.81 ng/ml to 10.67±1.50 ng/ml. MNA increased in the placebo group (n=3, slow metabolizer of caffeine) by 4.5%, from 6.87±1.13 ng/ml to 7.18±1.79 ng/ml.

### 50 mg Paraxanthine (Slow Metabolizer, n=3) vs. 50 mg Caffeine (Slow Metabolizer, n=3)

50 mg paraxanthine showed a 108% greater increase in MNA than 50 mg caffeine in slow metabolizer of caffeine.

### 200 mg Paraxanthine (Slow Metabolizer, n=3) vs. 200 mg Placebo (Slow Metabolizer, n=3)

Ingestion of 200 mg paraxanthine in slow metabolizer of caffeine (n=3) for 7 days significantly increased MNA by 45.5% from 9.26±0.50 ng/ml to 13.47±1.36 ng/ml. MNA decreased in the placebo group (n=3, slow metabolizer of caffeine) by 36.8%, from 6.34±1.26 ng/ml to 4.01±2.50 ng/ml.

### 200 mg Caffeine (Slow Metabolizer, n=3) vs. 200 mg Placebo (Slow Metabolizer, n=3)

Ingestion of 200 mg caffeine in slow metabolizer of caffeine (n=3) for 7 days increased MNA by 14.9% from 9.37±42.5 ng/ml to 10.77±1.49 ng/ml. MNA increased in the placebo group (n=3, slow metabolizer of caffeine) by 22.8%, from 5.40±4.39 ng/ml to 6.63±1.77 ng/ml.

### 200 mg Paraxanthine (Slow Metabolizer, n=3) vs. 200 mg Caffeine (Slow Metabolizer, n=3)

200 mg paraxanthine showed a 75% greater increase in MNA than 200 mg caffeine in slow metabolizer of caffeine.

### 50 mg Paraxanthine (Fast Metabolizer, n=3) vs. 50 mg Placebo (Fast Metabolizer, n=3)

Ingestion of 50 mg paraxanthine in fast metabolizer of caffeine (n=3) for 7 days did not change MNA levels: pre 10.34±2.20 ng/ml to post 10.34±1.58 ng/ml. MNA decreased in the placebo group (n=3, fast metabolizer of caffeine) by 58.2%, from 7.14±2.08 ng/ml to 2.98±1.96 ng/ml.

### 500 mg Caffeine (Fast Metabolizer, n=3) vs. 500 mg Placebo (Fast Metabolizer, n=3)

Ingestion of 200 mg caffeine in slow metabolizer of caffeine (n=3) for 7 days increased MNA by 5.4% from 9.03±3.53 ng/ml to 9.52±2.04 ng/ml. MNA decreased in the placebo group (n=3, slow metabolizer of caffeine) by 55.3%, from 8.67±0.90 ng/ml to 3.87±0.40 ng/ml. 200 mg paraxanthine (fast metabolizer, n=3) vs. 200 mg placebo (fast metabolizer, n=3)

Ingestion of 200 mg paraxanthine in fast metabolizer of caffeine (n=3) for 7 days significantly increased MNA by 33.8% from 8.56±1.72 ng/ml to 11.45±0.66 ng/ml. MNA increased in the placebo group (n=3, fast metabolizer of caffeine) by 1.4%, from 5.01±3.28 ng/ml to 5.08±2.04 ng/ml.

### 200 mg Caffeine (Fast Metabolizer, n=3) vs. 200 mg Placebo (Fast Metabolizer, n=3)

Ingestion of 200 mg caffeine in slow metabolizer of caffeine (n=3) for 7 days increased MNA by 31.2% from 7.70±1.95 ng/ml to 10.10±0.37 ng/ml. MNA decreased in the placebo group (n=3, slow metabolizer of caffeine) by 12.4%, from 6.28±1.58 ng/ml to 5.50±3.43 ng/ml.

200 mg paraxanthine (fast metabolizer, n=3) vs. 200 mg caffeine (fast metabolizer, n=3).

200 mg paraxanthine showed a 20.4% greater increase in MNA than 200 mg caffeine in fast metabolizer of caffeine.

Summary of Example 1 Results

| | All (N=6) | Slow Metabolizer (N=3) | Fast Metabolizer (N=3) |
|---|---|---|---|
| 50 mg Paraxanthine | +21.7% | +48.3 | 0% |
| 50 mg Placebo | -21.6% | +12.6% | -58.2% |
| 50 mg Caffeine | +13.6% | +22.2% | +5.4% |
| 50 mg Placebo | -28.9% | +4.5% | -55.3% |
| Increase with PX over CAF | +66.8 % | +108 % | |
| 200 mg Paraxanthine | +40.0% | +45.5% | +33.8% |
| 200 mg Placebo | -20.1 % | -36.8% | + 1.4% |
| 200 mg Caffeine | +22.2% | + 14.9% | +31.2% |
| 200 mg Placebo | +3.7% | +22.8% | -12.4% |
| Increase with PX over CAF | +86.9% | +75% | +20.4% |

### EXAMPLE 2 Paraxanthine vs. Caffeine in Slow and Fast Metabolizers of Caffeine Cognition, Memory, Learning (Brain-Derived Neurotrophic Factor)

### Methods

Brain Derived Neurotrophic Factor (BDNF) is a key molecule involved in plastic changes related to learning and memory (Miranda et al. Brain-Derived Neurotrophic Factor: A Key Molecule for Memory in the Healthy and the Pathological. Brain. Front. Cell. Neurosci. 2019, 13:363.). Increases in BDNF are linked to improved cognition.

### Study Design

### 1 Design

Randomized, double-blind, cross-over, four arm, four treatments, controlled study.

### 2 Number of Subjects

12 subjects with 6 subjects in each of the 2 arms in Period I. After a washout period of 2-weeks, same subjects participated again in Period II.

### 3 Randomization

Investigational products duly labeled with randomization codes were provided to the investigators by the sponsor. Per the randomization schedule the investigator / designee were dispense IPs on Day 0. The IPs was kept by the investigator in a safe but accessible place.

### 4 Supplementation

### Period I

Arm 1: Decaffeinated beverage plus 50 mg Caffeine (3 slow and 3 fast metabolizers)

Arm 2: Decaffeinated beverage plus 50 mg enfinityⓇ Paraxanthine (3 slow and 3 fast metabolizers)

Post completion of seven days treatment in Period I, subjects were asked to visit the site after 14 days of washout period. The same set of subjects participated in Period II. They were administered the following doses of investigational products;

### Period II

Arm 1: Decaffeinated beverage plus 50 mg Placebo (same group received 50 mg of caffeine in Period I) - same 6 subjects of Period I

Arm 2: Decaffeinated beverage plus 50 mg Placebo (same group received 50 mg of paraxanthine in Period I) - same 6 subjects of Period I

Each group of 6 subjects had a subset of 3 slow and 3 fast caffeine metabolizers. In total, all the 2 groups/arms had 6 slow caffeine metabolizers and 6 fast caffeine metabolizers. A total of 12 subject’s data was analyzed towards the end of the study.

### 5. Genomic Testing

The blood samples were collected and the genomic sequencing was analyzed, on the Basis of reports the subjects were divided into 2 groups having slower metabolism & faster metabolism.

### 6. Blood Sample

3 ml of blood was withdrawn in each visit from each subject and Brain-Derived Neurotrophic Factor (BDNF) as a biomarker of cognition was analyzed by LCMS.

### 7. Results

Paraxanthine significantly increased BDNF levels, showing a 37.5% greater increase of BDNF compared to caffeine. When analyzed for changes in BDNF levels in subgroups of fast and slow metabolizers of caffeine, paraxanthine showed a 47.3% greater increase in slow metabolizers in caffeine compared to caffeine.

Results Summary Table

| | All (N=6) | Slow Metabolizer (N=3) | Fast Metabolizer (N=3) |
|---|---|---|---|
| 50 mg Paraxanthine | +6.5% | +9.8% | +3.2% |
| 50 mg Placebo | -0.1% | +0.5% | -0.7% |
| 50 mg Caffeine | +4.8% | +6.7% | +3.0% |
| 50 mg Placebo | -1.2% | -0.4% | -1.9% |
| Increase with PX over CAF | +37.5% | +47.3% | +8.1% |

### EXAMPLE 3 Paraxanthine vs. Caffeine in Slow Metabolizers of Caffeine Sports Nutrition, Energy, Focus, Mood, Jitters

A healthy female subject, age range 31-45, classified as slow caffeine metabolizer through genomic testing, experiences by drinking coffee a jittery feel jittery and concentration issues like causing mental fog which causes the avoidance of coffee in the afternoon.

After talking 100 mg paraxanthine in capsule form in the morning the subject felt a noticeable increased level of energy, focus, and mood within 30 minutes, which had a long-lasting effect for up to 4 hours compared to the intake of caffeine. In addition, the emotional and physical activity levels were improved, resulting in faster running times (2.5 miles run) and increased distance of 3 miles through the improved great feeling during the exercise. The intake of a higher dosage of paraxanthine with 200 mg resulted in similar results without a negative impact.

### EXAMPLE 4 Paraxanthine vs. Caffeine in Slow Metabolizers of Caffeine Confidence, Sleep, Energy, Focus, Mood, Jitters

A healthy male subject, age range 31-45, classified as slow caffeine metabolizer through genomic testing, experiences by drinking coffee in low doses, <200 mg, that it provides energy but also jitters, anxiety, increased heart rate. Larger doses are more problematic in terms of anxiety, heart rate, and negatively affect the subject’s sleep, which results in the avoidance of drinking coffee in the afternoon.

After taking 100 mg paraxanthine in capsule form in the morning the subjects experienced noticeable improvements compared to caffeine in energy, focus, and mood, resulting in feeling confident, happy and alert, which allowed the subject to be more mindful and content from task to task. In addition, the intake of paraxanthine did not have a negative impact on the ability to fall asleep and stay asleep. These effects and experiences were even further improved with higher dosages of paraxanthine with 200 mg and 300 mg.

### EXAMPLE 5 Paraxanthine vs. Caffeine in Slow Metabolizers of Caffeine Anxiety, Energy, Focus, Mood, Jitters

A healthy female subject, age range 46-65, classified as slow caffeine metabolizer through genomic testing, experiences by drinking daily coffee in excess anxiety and jittery, worst case migraines, and anything after 11am interrupts the daily sleep patterns. As a result, the subjects can manage it by drinking ½ caffeine and ½ decaffeinate by volume with less negative effects.

After talking 200 mg paraxanthine in capsule form in the morning the subject felt a noticeable increased level of energy, focus, and mood within 30 minutes, which had a long-lasting effect for up to 4 hours compared to the intake of caffeine. The biggest difference the subject noticed is that it feels much cleaner. No signs of any anxiety or jittery-ness, and the mind feels really clear, and strong, improved levels of energy. The subjects experienced even a further improvement by using higher dosages with 200 mg and 300 mg paraxanthine.

### EXAMPLE 6 Fast vs. Slow Metabolizers of Caffeine--Cognition

Design: 6 slow metabolizers of caffeine were separated into 2 groups. Subjects completed the serial subtraction test (SST), in which they had to subtract the number 7 from 100, 14 consecutive times to evaluate cognitive performance. The total time each subject needed to complete the test was recorded, serving as a measure of cognitive performance. After baseline testing, the subjects were randomly appointed to consume either 200 mg of caffeine (female, age 46; female, age 31; male, age 30) or 200 mg of paraxanthine (male, age 30; female age 27, female, age 25) for 7 days and then repeated baseline testing.

Results: Consumption of 200 mg caffeine reduced the average time to complete the SST from 76.7 seconds at baseline to 74.7 seconds. Consumption of 200 mg paraxanthine, however, reduced the average time to complete the SST from 74.3 seconds at baseline to 70.0 seconds. The improvements in cognitive performance (2.0 seconds in the caffeine group, 4.3 seconds in the paraxanthine group) were 115% greater in the paraxanthine group when compared to the caffeine group in a group of slow metabolizers of caffeine.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

What is claimed is:

1. A method for improving cognitive function in a slow caffeine metabolizer (SCM) subject, comprising:
a. identifying an individual as a SCM subject; and
b. providing the SCM subject with a composition comprising about 2 mg to about 800 mg of paraxanthine.

2. The method of claim 1, wherein the step of identifying the SCM subject comprises genotyping the individual for a genetic variant associated with slow caffeine metabolism.

3. The method of claim 2, wherein the genetic variant is in a CYP1A2, ADORA2A and/or CYP2E1 gene.

4. The method of claim 3, wherein the genetic variant is in the CYP1A2 gene and the subject’s genotype is AC or CC.

5. The method of claim 2, wherein the SCM subject is identified by administering the individual a questionnaire on the individual’s response to caffeine.

6. The method of claim 1, wherein the SCM subject self-diagnoses as SCM.

7. The method of claim 1, wherein the step of identifying the SCM subject comprises measuring the individual’s rate of caffeine metabolism.

8. The method of claim 1, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional setshifting, delayed reinforcement learning, reversal learning, the temporal integration of voluntary behavior, speed of processing, reasoning, problem solving and/or social cognition in the SCM subject.

9. The method of claim 1, wherein the composition further comprises at least one agent selected from 1-methylxanthine and/or 7-methylxanthine.

10. The method of claim 9, wherein the 1-methylxantine and/or 7-methylxantine are present in the composition in an amount from about 50 mg to about 400 mg.

11. The method of claim 1, wherein administration of the composition to the SCM subject increases serum Brain Derived Neurotrophic Factor (BDNF) concentration of at least about 10% relative to a fast caffeine metabolizer subject who received a comparable dose of the composition.

12. The method of claim 1, wherein the composition further comprises at least one ingredient selected from the group consisting tyrosine, N-acetyl-tyrosine, taurine, huperzine A, acetyl-1-carnitine, CDP choline, Alpha GPC, choline bitrate, choline citrate, B 12, caffeine, methyllliberine, theacrine, paraxanthine, theobromine, ashwagandha, rhodiola, lutein, zeaxanthin, fish oil, creatine, ginseng, lions mane, niacin, cordyceps, theanine, B-vitamins, GABA, sulbutiamine, vinpocetine, adenosine triphosphate, inositol, enhanced arginine silicate, nitrates, electrolytes, hesperidin and derivatives of hesperidin and/or bacopa.

13. A method for improving athletic performance in a SCM subject in comprising administering to the subject a composition comprising an effective amount of paraxanthine.

14. The method of claim 13, wherein the amount of paraxanthine is administered is from about 50 mg to about 400 mg.

15. The method of claim 14, wherein the subject experiences and increase in endurance.

16. The method of claim 13 wherein the composition further comprises 1-methylxanthine in an amount from about 2 mg to about 800 mg and wherein administration of the composition to a subject produces a synergistic increase in athletic performance to the administration of a comparable dose of paraxanthine or 1-methylxanthine alone.

17. The method of claim 13, wherein administration of the composition to the SCM subjectincreases serum N-methylnicotinamide (MNA) concentration from about 20-100% relative to the change in the subject following administration of a comparable dose of caffeine.

18. A method of promoting weight loss in a SCM subject comprising:
a. identifying an individual as a SCM subject; and
b. providing the SCM subject with a composition comprising about 2 mg to about 800 mg of paraxanthine.

19. The method of claim 18, wherein weight loss is promoted through inducing thermogenesis in the subject.

20. The method of claim 19, wherein the composition further comprises one or more compounds selected from a list consisting of: caffeine, green tea, capsaicin, garcinia cambogia, yohimbine and bitter orange.

21. The method of claim 18, wherein administration of the composition to the SCM subject increases serum N-methylnicotinamide (MNA) concentration from about 20-100% relative to the change in the subject following administration of a comparable dose of caffeine and wherein the subject experiences an increased perception of energy and/or a decreased perception of jitteriness.

22. The method of claim 18, wherein weight loss is promoted through enhancing lipolysis in the subject.
